(19) United States
(12) Patent Application Publication
(10) Pub. No.: US 2009/0247407 A1
(43) Pub. Date: Oct. 1, 2009

(54) Glyceride Ester Derivatives of Herbicidal Compounds and Compositions Thereof

(75) Inventors: Edward Kravets, Victoria (AU); Phillip Hay, Victoria (AU); Graeme Sutton, Victoria (AU)

Correspondence Address: NORRIS, MCLAUGHLIN & MARCUS, 875 THIRD AVE, 18TH FLOOR, NEW YORK, NY, 10022, US

(73) Assignee: Nufarm Australia Limited, Laverton North, Victoria (AU)

(21) Appl. No.: 12/295,928

(22) PCT Filed: Apr. 5, 2007
(86) PCT No.: PCT/AU2007/000442

§ 371 Date: Dec. 12, 2008

Related U.S. Application Data
(60) Provisional application No. 60/810,633, filed on Jun. 5, 2006.

(30) Foreign Application Priority Data
Apr. 5, 2006 (AU) 2006901790

(51) Int. Cl. A01N 57/18; C07C 69/76; A01N 37/00; A01P 13/00

(52) U.S. Cl. 504/206; 560/55; 504/317

(57) ABSTRACT

A herbicidal compound and composition comprising at least one compound of formula I wherein R is independently selected from hydrogen, and the group (CH₂)ₘOR⁴; m, q, t and v are independently zero or one; R¹, R², R³ and R⁴ are independently selected from the group consisting of hydrogen, lower alkyl (preferably C₁ to C₄ alkyl) and lower acyl (preferably C₂ to C₄ acyl) and the group of formula II wherein the compound of formula I comprises at least one group R¹, R², R³ and R⁴ which is of formula II wherein A is N or CH; X is selected from the group consisting of chloro, amino, methyl and methoxy; X¹ is selected from the group consisting of hydrogen, chloro and methyl; X² is hydrogen or chloro; R¹ is selected from hydrogen and methyl; Y is oxygen or a bond; and n is from 0 to 3.

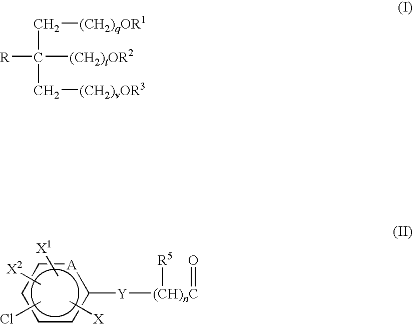

### FIELD

This invention relates to herbicidal compounds, compositions containing the compounds and a method of controlling weeds.

### BACKGROUND

Aryl acid and aryloxy acid herbicides have been in use for many years. Phenoxy acid herbicides include phenoxy-acetic, propionic and butyric acid herbicides and their esters. Phenoxy acetic acid herbicides including 2,4-dichlorophenoxy acetic acid (2,4-D) and 4-chloro-2-methylphenoxy acetic acid (MCPA) and their esters such as the ethyl, 2-ethylbutyl and butoxyethyl and 2-ethylhexyl esters are used to control broadleaf weeds in crops such as cereals, sugar cane turf pastures and the like. Benzoic acid herbicides such as dicamba, the pyridyloxy herbicides such as triclopyr and pyridine carboxylic acids such as picloram are an analogous group. Volatile esters such as the ethyl ester are not recommended for use in susceptible crops as the vapors may effect plants in the immediate vicinity of spray application. This has lead to the higher esters being favored for some applications. Also the volatility of the esters from soil and plant foliage is often quite different. For example while the ethyl ester is volatilized more readily from plant foliage the isobutyl ester is more readily volatilized from soil than from foliage.

Volatilization is the process by which a compound evaporates in the vapour phase to the atmosphere from another environmental compartment and is an important mechanism for the loss of pesticides, after application, from plant surfaces and soils to the atmosphere, with potential subsequent movement to off-target areas.

Concern regarding off-target damage caused by vapour drift for aryl and aryloxy ester herbicides with high volatility has lead to regulatory restriction on the use of these esters; however the high herbicidal efficacy of phenoxy ester herbicides, compared to water soluble salts of phenoxy herbicides, is desirable to reduce the total quantity phenoxy herbicides applied.

Aryl ester aryloxy ester herbicides are generally formulated as solutions in organic solvents and more specifically as emulsion concentrates or concentrated emulsions. They may also be absorbed on a carrier. The formulation of aryl acid and aryloxy acid herbicides in solid forms such as suspension concentrates, water dispersible powders and water dispersible granules has been limited by the difficulty faced in milling the compounds which are generally waxy or relatively low melting solids. It is desirable to provide a solid formulation and method of preparing same to extend the formulation options of this important class of herbicides. This would also allow higher loadings of the herbicides to be transported thereby reducing transport costs and improving handling by the manufacturer and farmer.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of this application.

### SUMMARY

The invention provides a herbicidal composition comprising at least one compound of formula I

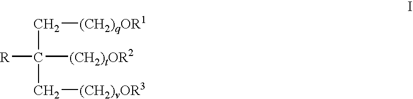

wherein
R is independently selected from hydrogen and the group (CH₂)ₘOR⁴;
m, q, t, and v (which are each independently selected) are each zero or one;
R¹, R², R³ and R⁴ are independently selected from the group consisting of hydrogen, lower alkyl (preferably C₁ to C₄ alkyl) and lower acyl (preferably C₂ to C₄ acyl) and the group of formula II wherein the compound of formula I comprises at least one group R¹, R², R³ and R⁴ which is of formula II

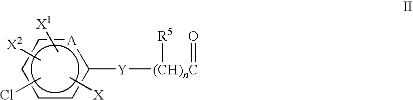

wherein
A is N or CH;
X is selected from the group consisting of chloro, amino, methyl and methoxy;
X¹ is selected from the group consisting of hydrogen, chloro and methyl;
X² is hydrogen or chloro;
R⁵ is selected from hydrogen and methyl;
Y is oxygen or a bond; and
n is from 0 to 3.

The group of formula II includes the group IIa

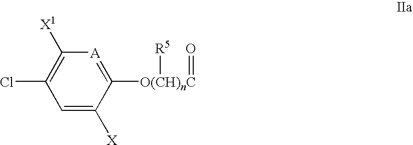

wherein
A is N or CH and is preferably CH;
X is selected from the group of halogen (preferably chloro) and methyl, preferably chloro and methyl and most preferably chloro;
X¹ is selected from hydrogen, halogen (preferably chloro) and methyl, preferably from hydrogen and chloro and most preferably is hydrogen; and
n is from 1 to 3.

The compounds of formula I also include the group of compounds of formula IIb

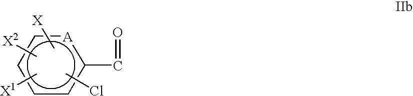

wherein
A is N or CH;
X is selected from the group consisting of chloro, amino, methyl and methoxy;
X¹ is selected from the group consisting of hydrogen, chloro and methyl; and
X² is hydrogen or chloro.

The acid (ie o-acyl) portion or portions of the compound of formula I are preferably derived from a herbicidal acid selected from the group consisting of:

| | |
|---|---|
| 2,4-D | (2,4-dichlorophenoxyacetic acid), |
| 2,4-DB | 2-(2,4-dichlorophenoxy)butyric acid, |
| dichlorprop | (RS)-2-(2,4-dichlorophenoxy)propionic acid, |
| dichlorprop-P | (R)-2-(2,4-dichlorophenoxy)propionic acid, |
| MCPA | 4-chloro-o-tolyloxyacetic acid, |
| MCPB | 4-(4-chloro-0-tolyloxy)butyric acid, |
| mecoprop | (RS)-2-(4-chloro-o-tolyloxy)propionic acid, |
| mecoprop-P | (R)-2-(4-chloro-o-tolyloxy)butyric acid and |
| triclorpyr | 3,5,6-trichloro-2-pyridyloxyacetic acid. |
| dicamba | 3,6-dichloro-o-anisic acid |
| picloram | 4-amino-3,5,6-trichloropyridine-2-carboxylic |
| | acid |
| clopyralid | 3,6-dichloropyridine-2-carboxylic acid |
| fluroxypyr | 4-amino-3,5-dichloro-6-fluoro-2- |
| | pyridyloxyacetic acid |
| aminopyralid | 4-amino-3,6-dichloro-2-pyridinecarboxylic |
| | acid |

The composition of the invention will preferably contain a carrier. The composition may be in the form of a concentrate for dilution (for example dilution with water) or it may be in the form of a dilute composition ready for application to plants.

The invention further provides a method of controlling weeds comprising application to the weeds or locus thereof of a composition as described above. The composition may be used for control of weeds in both crop and non-crop situations.

The compounds of formula I include compounds of formula

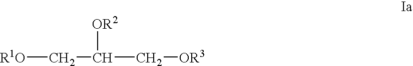

wherein at least one of R¹, R² and R³ is of formula II; and compounds of formula I

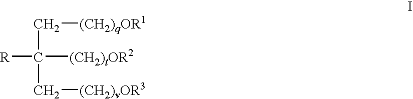

wherein
m, q, t, and v, R, R¹, R² and R³ are as hereinbefore defined and at least one of m is one;
R¹, R², R³ and m are as hereinbefore defined and when R is hydrogen at least one of m, q, t, v is at least one.

Certain compounds of formula I are new compound.

In another embodiment the invention therefore provides compounds of formula I

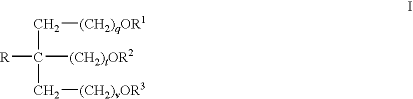

wherein
R is hydrogen or (CH₂)ₘOR⁴;
R¹, R², R³ and m, q, t and v are as hereinbefore defined and when R is hydrogen at least one of q, t and v is one.

In one preferred embodiment the a herbicidal composition comprises at least one compound of formula III

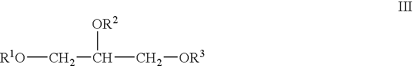

wherein at least one of R¹, R² and R³ is a group of formula IV

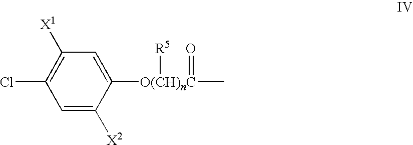

wherein
   X¹ is selected from hydrogen, halogen (preferably chloro) and methyl, preferably from hydrogen and chloro and most preferably is hydrogen;
   X² is selected from the group of hydrogen, halogen (preferably chloro) and methyl, preferably chloro and methyl and most preferably chloro;
   R⁵ is selected from hydrogen and methyl and preferably is hydrogen; and
   n is from 1 to 3.

The groups R¹, R² and R³ which are not of formula II are preferably selected from hydrogen and aliphatic such as lower aliphatic (e.g. C₁ to C₄ aliphatic).

Most preferably those groups R¹, R² and R³ which are not of formula II are each hydrogen.

The compound of formula I may be in the form of the mono glyceride, diglyceride, triglyceride of the phenoxy acid group or a mixture of two or more of the mono-, di- and tri-glycerides may be present.

The composition of the invention will preferably contain a carrier. The composition may be in the form of a concentrate for dilution (for example dilution with water) or it may be in the form of a dilute composition ready for application to plants.

The compounds of the invention generally have a softening point significantly higher than the currently used aryl ester and aryloxy ester herbicides enabling them to be processed and handled in solid form. They may be processed to achieve particle size reduction to form fine particles thus making the compounds particularly suited to storage and handling in solid formulations such as in powders, suspension concentrates, prills and water dispersible granules.

Accordingly the invention also provides a solid particulate composition comprising the above described compounds wherein the compounds in solid form have been subject to particle size reduction.

In a preferred embodiment the invention provides a herbicidal composition comprising at least one of the compounds of formula I in solid particulate form preferably selected from the group consisting of powders, suspensions such as suspension concentrates, prills and water soluble granules.

The preferred compositions will generally be prepared by milling the solid compounds to reduce the particle size so that at least 90% by weight is no more than 1000 microns preferably no more than 500 microns and most preferably at least 90% by weight of the composition is of particle size no more than 200 microns. Generally the particles will be of at least 10 microns in size.

The invention further provides a process for preparing a particulate aryl ester or aryloxy ester herbicide comprising providing the ester of formula I in solid form, milling the solid to form a solid particulate herbicide; and optionally formulating the particulate solid with one or more selected from carriers, adjuvants and fillers.

The solid may be milled in the presence of other materials which may be liquid or solid. Milling in the presence of an oil or surfactant may aid in forming a stable dispersion and/or inhibiting agglomeration of the solid.

The invention further provides a method of controlling weeds comprising application to the weeds or locus thereof of a composition as described above. The composition may be used for control of weeds in crops.

Throughout the description and the claims of this specification the word “comprise” and variations of the word, such as “comprising” and “comprises” is not intended to exclude other additives, components, integers or steps.

### DETAILED DESCRIPTION

The invention provides a herbicidal composition comprising at least one compound of formula I

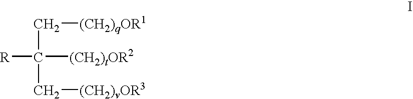

wherein
R is independently selected from hydrogen and the group (CH₂)ₘOR⁴; each of m, q, t and v (which are each independently selected) is 0 or 1;
R¹, R², R³ and R⁴ are independently selected from the group consisting of hydrogen, lower alkyl (preferably C₁ to C₄ alkyl) and lower acyl (preferably C₂ to C₄ aryl) and the group of formula II wherein the compound of formula I comprises at least one group R¹, R², R³ and R⁴ which is of formula II

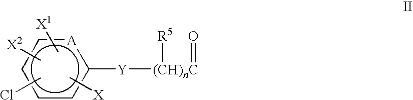

wherein
A is N or CH;
X is selected from the group consisting of chloro, amino, methyl and methoxy;
X¹ is selected from the group consisting of hydrogen, chloro and methyl;
X² is hydrogen or chloro;
R⁵ is selected from hydrogen and methyl;
Y is oxygen or a bond; and
n is from 0 to 3.

Preferably at least two of R¹, R², R³ and R⁴ are of formula II and most preferably at least three are of formula II.

The group of formula II includes the group IIa

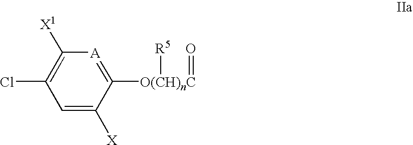

wherein
A is N or CH and is preferably CH;
X is selected from the group of halogen (preferably chloro) and methyl, preferably chloro and methyl and most preferably chloro;
X¹ is selected from hydrogen, halogen (preferably chloro) and methyl, preferably from hydrogen and chloro and most preferably is hydrogen; and
n is from 1 to 3.

The compounds of formula II also include the group of compounds of formula IIb

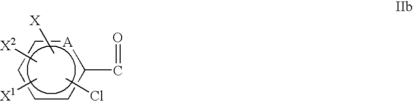

wherein
A is N or CH;
X is selected from the group consisting of chloro, amino, methyl and methoxy;
X¹ is selected from the group consisting of hydrogen, chloro and methyl; and
X² is hydrogen or chloro.

The acid (ie o-acyl) portion or portions of the compound of formula I are preferably derived from a herbicidal acid selected from the group consisting of:

| | |
|---|---|
| 2,4-D | (2,4-dichlorophenoxyacetic acid), |
| 2,4-DB | 2-(2,4-dichlorophenoxy)butyric acid, |
| dichlorprop | (RS)-2-(2,4-dichlorophenoxy)propionic acid, |
| dichlorprop-P | (R)-2-(2,4-dichlorophenoxy)propionic acid, |
| MCPA | 4-chloro-o-tolyloxyacetic acid, |
| MCPB | 4-(4-chloro-0-tolyloxy)butyric acid, |
| mecoprop | (RS)-2-(4-chloro-o-tolyloxy)propionic acid, |
| mecoprop-P | (R)-2-(4-chloro-o-tolyloxy)butyric acid and |
| triclorpyr | 3,5,6-trichloro-2-pyridyloxyacetic acid. |
| dicamba | 3,6-dichloro-o-anisic acid |
| picloram | 4-amino-3,5,6-trichloropyridine-2-carboxylic |
| | acid |
| clopyralid | 3,6-dichloropyridine-2-carboxylic acid |
| fluroxypyr | 4-amino-3,5-dichloro-6-fluoro-2- |
| | pyridyloxyacetic acid |
| aminopyralid | 4-amino-3,6-dichloro-2-pyridinecarboxylic |
| | acid |

In a particularly preferred embodiment the compounds comprise at least two such ester groups. In the case of glycerol the preferred compounds comprise two such ester groups and in a further preferred embodiment the compounds comprise three such ester groups (i.e. they are triglycerides of one or more of the herbicidal acid groups) In the case of pentaerythritol the number of ester groups may be one, two, three or four or the composition may contain a mixture of two or three herbicidal acid derived ester groups.

The invention provides in one embodiment a herbicidal composition comprising at least one compound of formula III

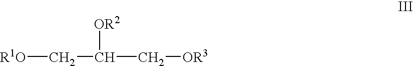

wherein at least one of R¹, R² and R³ is a group of formula IV

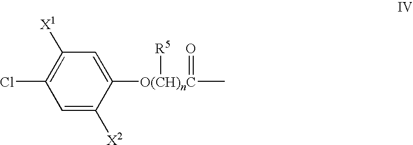

wherein
   X¹ is selected from hydrogen, halogen (preferably chloro) and methyl, preferably from hydrogen and chloro and most preferably is hydrogen;
   X² is selected from the group of hydrogen, halogen (preferably chloro) and methyl, preferably chloro and methyl and most preferably chloro;
   R⁵ is selected from hydrogen and methyl and preferably is hydrogen; and
   n is from 1 to 3 and it is particularly preferred that n is 1; and groups R¹, R² and R³ which are not of formula II are independently selected from hydrogen and alkyl such as lower alkyl (e.g. C₁ to C₆ alkyl) and fatty alkyl (e.g. C₆ to C₂₀ alkyl); and
      a carrier, preferably a carrier comprising a surfactant and more preferably also a non-volatile solvent.

Preferably at least two of R¹, R² and R³ are of formula IV.

The alcohols which may be used in preparing compounds of formula I include alcohols of formula VI:

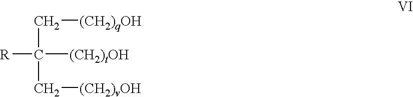

wherein R is hydrogen or (CH₂)ₘOH the values of m, q, t and v are independently 0 or 1.

Specific examples of alcohols of formula VI where R is hydrogen which are useful in preparing compounds of formula I include glycerol, butan-1,2,4-triol, 2-(hydroxymethyl)butan-1,4-diol, 3-(hydroxymethyl)pentan-1,5-diol, pentan-1,3,5-triol. Specific examples of alcohols of formula VI where R is (CH₂)_{q}OR⁴ include 2-(hydroxymethyl)propan-1,2,3-triol, 2-(hydroxymethyl)butan-1,2,4-triol, 2,2-bis(hydroxymethyl)butan-1,4-diol, 3,3-bis(hydroxymethyl)pentan-1,5-diol, 3-(hydroxylmethyl)pentan-1,3,5-triol and pentaerythritol.

The corresponding esters from alcohols of formula VI include the mono, di and tri herbicidal acid derived esters of glycerol; the mono, di and tri-herbicidal acid esters of butan-1,2,4-triol; the mono-, di- and tri-herbicide acid esters of 2-hydroxymethyl)butan-1,4-diol; the mono-, di- and tri herbicide acid esters of 3-(hydroxymethyl)pentan-1,5-diol; and the mono-, di- and tri-esters of pentan-1,3,5-triol.

The corresponding herbicidal acid esters from alcohols of formula VI include the mono-, di-, tri- and tetra-herbicidal acid esters of 2-(hydroxymethyl)propan-1,2,3-triol, the mono-, di-, tri- and tetra-herbicidal acid esters of 2-(hydroxymethyl)butan-1,2,4-triol, the mono-, di-, tri- and tetra-herbicidal acid esters of 2,2-bis(hydroxymethyl)butan 1,4-diol, the mono-, di-, tri- and tetra-herbicidal acid esters of 3,3-bis(hydroxymethyl)pentan-1,5-diol, the mono-, di-, tri- and tetra-herbicidal acid esters of 3-(hydroxylmethyl)pentan-1,3,5-triol and the mono-, di-, tri- and tetra-herbicidal acid esters of pentaerythritol. The esters of pentaerythritol are particularly preferred esters of formula I and in particular the tri and tetra esters of pentaerythritol.

The compounds of formula I are preferably the monoesters or diesters of the alcohols and the monoesters are particularly preferred although in specific cases the di- or higher esters may be prepared and have the advantage of providing a higher loading of the acyl group of formula II on the basis of the weight of the compound of formula I.

The compounds of formula I may be prepared by a range of esterification method known in a general sense for preparation of esters of aromatic substituted esters or aryloxy substituted esters.

In one embodiment the invention provides a method of preparation of a compound of formula I for use in the compositions of the invention, the process comprising reacting an alcohol of formula I wherein R¹, R², R³ and R⁴ are selected from the group consisting of hydrogen, lower alkyl (preferably C₁ to C₄ alkyl), lower acyl (preferably C₂ to C₄ acyl) and at least one of R¹, R², R³ and R⁴ present in the compound of formula I is hydrogen with an acid or derivative thereof of formula V:

Y—R⁶ V

wherein Y is the group of formula II and R⁶ is —OH or a leaving group such as halogen or acyl optionally in the presence of a catalyst to provide a compound of formula I wherein at least one of R¹, R², R³ and R⁴ is of formula II.

Compounds comprising groups of formula II which are more specifically of formula IIa or IIb may similarly be prepared using the appropriate acid or acid chloride reactant of formula V wherein Y is of formula IIa or Iib and R⁶ is chloro.

The process of the invention may for example involve reacting the acid chloride of formula V wherein R⁶ is chloro with the alcohol of formula I wherein at least one of R¹, R², R³ and R⁴ is hydrogen, preferably at an elevated temperature of for example 300 to 150° C. to give the herbicidal compound of formula I.

Alternatively the acid of formula V (wherein R⁶ is —OH) may be reacted with the alcohol of formula I wherein at least one of R¹, R², R³ and R⁴ is hydrogen, (preferably at an elevated temperature of for example 300 to 180° C.), more preferably 700 to 150° C.) to give the herbicidal compound of formula I.

The compounds of the invention may be in the form of mixtures of isomers brought about by a plurality of reactive hydroxyl groups. Furthermore in some cases the compounds of formula I are optically active may be present as a racaemic mixture or may be enriched in one enantiomer for example it may be present as 60% or more (e.g. 80%) or more of one enantiomer (eg about 60%, more preferably at least 80% of one of the D and L optical isomers).

Specific examples of monoester compounds of the invention include the following (which may be present in admixture with other compounds of formula I of the same or different formula):
2,3-dihydroxy-1-propyl 2,4-dichlorophenoxyacetate,
3,4-dihydroxy-1-butyl 2,4-dichlorophenoxyacetate,
3-hydroxymethyl-4-hydroxy-1-butyl 2,4-dichlorophenoxyacetate,
2,3-dihydroxy-2-hydroxymethyl-1-propyl 2,4-dichlorophenoxyacetate,
3,3-di(hydroxymethyl)-4-hydroxy-1-butyl 2,4-dichlorophenoxyacetate,
3,5-dihydroxy-3-hydroxymethyl-1-pentyl 2,4-dichlorophenoxyacetate,
2,2-di(hydroxymethyl)-3-hydroxy-1-propyl 2,4-dichlorophenoxyacetate,
2,3-dihydroxy-1-propyl 2-(2,4-dichlorophenoxy)butyrate,
2,2-di(hydroxymethyl)-3-hydroxy-1-propyl 2-(2,4-dichlorophenoxy)butyrate,
2,3-dihydroxy-1-propyl(RS)-2-(2,4-dichlorophenoxy)propionate,
2,2-di(hydroxymethyl)-3-hydroxy-1-propyl(RS)-2-(2,4-dichlorophenoxy)propionate 4-chloro-o-tolyloxyacetic acid,
2,3-dihydroxy-1-propyl 4-chloro-o-tolyloxyacetic acid,
2,2-di(hydroxymethyl)-3-hydroxy-1-propyl (RS)-2-(4-chloro-o-tolyloxy)propionate,
2,3-dihydroxy-1-propyl (RS)-2-(4-chloro-o-tolyloxy)propionate,
2,2-di(hydroxymethyl)-3-hydroxy-1-propyl 3,5,6-trichloro-2-pyridyloxyacetate,
2,3-dihydroxy-1-propyl 3,5,6-trichloro-2-pyridyloxyacetate,
2,2-di(hydroxymethyl)-3-hydroxy-1-propyl 3,6-dichloro-o-anisate,
2,3-dihydroxy-1-propyl 3,6-dichloro-o-anisate,
2,2-di(hydroxymethyl)-3-hydroxy-1-propyl 6-dichloropyridine-2-carboxylate,
2,3-dihydroxy-1-propyl,6-dichloropyridine-2-carboxylate,
2,2-di(hydroxymethyl)-3-hydroxy-1-propyl 4-amino-3,5-dichloro-6-fluoro-2-pyridyloxyacetate and
2,3-dihydroxy-1-propyl 4-amino-3,5-dichloro-6-fluoro-2-pyridyloxyacetate.
2,3-dihydroxy-1-propyl 4-amino-3,6-dichloro-2-pyridinecarboxylate.

The compounds of the invention also include the corresponding di- and tri-esters.

Despite the enormous contribution phenoxy esters have made to farming over a period of over forty years herbicidal compositions of the compounds of formula I and their properties have, to our knowledge never been examined.

The compounds of the invention generally have a combination of low volatility from each of soil and plant foliage thus providing a margin of safety for non-target plants and crops which is greater than for most of the currently used phenoxy acid herbicidal esters. The compositions also allow a higher loading of herbicide in the concentrate than is often available for many of the commercially available esters. This balance of desirable properties together with the ready availability of suitable raw materials for manufacture provide significant advantages for the mono, di-, tri- and tetra esters when compared with the esters currently used in herbicidal compositions.

In some instances it is preferred that the compositions of the invention comprise a mixture of esters including at least 10% of the total herbicidal acyl group in the form of a second ester. Such mixtures may have favourable physical properties for formulation.

At least one of the groups R¹, R², R³ and R⁴ (when R⁴ is present) in the compound of formula I is of formula II. Preferably one or two of these groups is of formula II and most preferably three or four groups are of formula II.

The particularly preferred compounds of the invention are selected from the group consisting of the mono-, di-, tri- and tetra 2,4-D esters and the mono-, di-, tri- and tetra-MCPA of the alcohols of formula VI. of MCPA. The tri 2,4-D and Tri-MCPA esters of polyols such as glycerol are particularly preferred. The tetrakis 2,4D and tetrais MCPA esters of pentaerythritol are also preferred.

Referring to the attached drawing FIG. 1 is a bar chart comparing the volatilization flux from plant and soil of previously reported esters of 2,4-D with the mono-, di- and tri-glycerides of 2,4-D. The volatilization flux may be calculated using the model reported by Woodrow, J. E. and Seiber, J. N. (Environ. Sci. Technol. 31, 523-527, 1997).

### BRIEF DESCRIPTION OF THE DRAWINGS

FIG. 1 shows that whereas the other esters have a significant and likely measurable volatilization flux the glycerol esters have a significantly lower and negligible or near zero flux from soil and from plant foliage.

FIG. 2 is a column chart showing the response Canola Brassica napus to application of a 2,4-D ester in accordance with the invention.

FIG. 3 is a column chart showing the response of Variegated Thistle Silybum marianum to application of a 2,4-D ester in accordance with the invention.

The invention further provides a compound of formula I wherein at least two groups selected from R, R¹, R² and R³ and are of formula II.

The composition of the invention is preferably in the form of a concentrate typically containing at least 100 grams per Litre of compound of formula I (based on corresponding acid equivalent), preferably 300 grams per Litre of compound of formula I (based on corresponding acid equivalent) more preferably at least 400 g/L still more preferably at least 500 g/L and most preferably at least 600 g/L (based on corresponding acid equivalent). Thus in the case of the 2,4-D the composition preferably contains 300 g/L more preferably at least 400 g/L still more preferably 500 g/L and most preferably at least 600 g/L based on the corresponding 2,4-D acid equivalent of the 2,4-D in esterified form.

Examples of preferred solid types of herbicidal compositions according to the present invention (in which amounts are expressed on a weight basis) are as follows:
(i) aqueous suspension concentrates which comprise from 5 to 70% by weight of compounds of formula I, from 2 to 15% of surfactant;
(ii) wettable powders which comprise from 5 to 90% of compounds of formula I, from 2 to 20% e.g. 5 to 15% surfactant and from 8 to 88% of solid diluent or carrier;
(iii) water dispersible granules which comprise from 1 to 90%, e.g. 25 to 75% of compounds of formula I, from 1 to 15%, e.g. 2 to 10%, of surfactant and from 0 to 95% e.g. 5 to 95%, more preferably 20 to 60%, of solid diluent, e.g. clay, granulated with the addition of water to form a paste and then dried;
(iv) water soluble or water dispersible powders which comprise from 5 to 90% compounds of formula I, from 2 to 30% of surfactant and from 0 to 88% of solid diluent; and
(v) liquid emulsifiable suspension concentrates which comprise from 5 to 70% compounds of formula I, from 5 to 15% of surfactant and from 0.1 to 5% of thickener and from 10 to 84% of organic solvent, e.g. mineral oil.

The invention also provides a herbicidal composition comprising:
(1) one or more phenoxy acid esters of formula I,
(2) a non-volatile solvent,
(3) at least one surfactant, and
(4) optionally customary auxiliaries and additives, such as thickeners and thixotropic agents, wetting agents, anti-drift agents, adhesives, penetration agents, preservatives and frost protection agents, antioxidants, fillers, carrier substances, dyes, fragrances, antifoams, fertilizers, evaporation inhibitors, and agents which influence the pH and the viscosity.

Examples of herbicidal compositions according to the present invention (in which amounts are expressed on a weight basis) are as follows:
(i) liquid water miscible concentrates which comprise from 5 to 50%, e.g. 10 to 50% of compound(s) of formula I, 5 to 25% of surfactant and from 10 to 90%, e.g. 45 to 85%, of water miscible solvent composition, or a mixture or water-miscible solvent and/or water; and
(ii) emulsifiable concentrates which comprise 0.05 to 90%, and preferably from 1 to 60% of compound(s) of formula I, from 0.1 to 20% surfactant, and preferably from 39 to 98.99%, of organic solvent.

The herbicidal composition of the present invention is preferably an emulsifiable concentrate. The composition is preferably a solution which on dilution with water forms an emulsion of the organic phase in water which is sufficiently stable to allow application of the diluted composition to the site of use. It is particularly preferred that the composition comprise at least one emulsifier adapted to provide an oil-in-water emulsion on dilution of the concentrate with water prior to use.

When used solvents in the composition of the invention are preferably non-volatile. Non-volatile solvents will typically have a flash point of greater than 60.5° C. and more preferably at least 65° C. Examples of suitable solvents are non-polar water-immiscible solvents or polar aprotic water miscible organic solvents. The non-polar solvents are selected from the group consisting of aliphatic or aromatic hydrocarbons and esters of plant oils or mixtures thereof.

Further examples of carrier solvents include acetophenone, cyclohexanone, isophorone, alkyl acetate esters such as ethyl, propyl and butyl acetates and mineral, animal, and vegetable oils (these diluents may be used alone or in combination). The preferred organic solvent comprises at least one hydrocarbon selected from alkyl substituted aromatics such as mono-, di- and trialkyl benzenes and alkyl naphthalenes. A person skilled in the art will readily be able to formulate suitable solvents and combinations to provide suitable solvent components on the basis of the teaching herein and widely reported flash point information. For example C₉ alkylbenzene is reported to have a flash point of 42° whereas C₁₀ alkylbenzene is reported to have a flash point of 66° C. A preferred co-solvent is a mixture of C₈-C₁₂ di- and trialkyl benzenes.

Esters of plant oils, which are used as nonpolar, water-immiscible solvents or adjuvants particularly in solid formulations according to the present invention, are typically alkyl esters obtainable from medium chained fatty acids by esterification with alkanols or by transesterification of the corresponding plant oils preferably in the presence of a lipase. Preferred fatty acids of these plant oils have 5 to 20, in particular 6 to 15 carbon atoms. In a preferred embodiment, the methyl ester of the plant oil used is the methyl ester of caprylic/capric ester or of capric ester having a distribution of fatty acid chain lengths around 10 units.

The water-miscible polar aprotic organic solvents are preferably compounds which exhibit a dielectric constant of 2.5 or more at 25° C., in particular from 2.7 to 4.0 at 25° C. Particularly preferred are cyclic amides and lactones as for example N-methylpyrrolidone, N-cyclohexylpyrrolidone and γ-butyrolactone, most preferred are γ-butyrolactone and N-methylpyrrolidone or mixtures thereof.

Also preferred are water-miscible polar aprotic solvents selected from the group consisting of alkyl lactates, in particular, isopropyl lactate, alkyl carbonates, alkylene carbonates, polyethylene glycols, polyethylene glycol alkyl ethers, polypropylene glycols and polypropylene glycol alkyl ethers, and most preferably particular isopropyl lactate, or mixtures thereof.

One class of classes of solvents which may be used in compositions of the invention is of formula (VII):

R⁵—CO—NR⁶R⁷ (VII)
wherein
R⁵, R⁶ and R⁷ are alkyl.

Preference is given to solvents of the formula (VII) in which R⁵=ethyl or propyl, such as n-propyl or isopropyl, and R⁶ and R⁷ are identical or different, preferably identical, and are (C₁-C₆)alkyl, e.g. methyl, ethyl, propyl, such as n-propyl or isopropyl, butyl, such as n-butyl, or branched butyl, such as sec-butyl, isobutyl or tert-butyl, pentyl, such as n-pentyl or branched pentyl, such as isopentyl or neopentyl, hexyl, such as n-hexyl or branched hexyl.

Examples of alkyl radicals R⁶ and R⁷ are methyl, ethyl, propyl, such as n-propyl or isopropyl, butyl, such as n-butyl, or branched butyl, such as sec-butyl, isobutyl or tert-butyl, pentyl, such as n-pentyl or branched pentyl, such as isopentyl or neopentyl, hexyl, such as n-hexyl or branched hexyl, heptyl, such as n-heptyl or branched heptyl, octyl, such as n-octyl or branched octyl, nonyl, such as n-nonyl or branched nonyl, decyl, such as n-decyl or branched decyl, undecyl, such as n-undecyl or branched undecyl, dodecyl, such as n-dodecyl or branched dodecyl. In a preferred embodiment, alkyl radicals R⁶ and R⁷ are identical.

Examples of such solvents include, for example, N,N-di-tert-butylformamide, N,N-dipentylformamide, N,N-dihexylformamide, N,N-diheptylformamide, N,N-dioctylformamide, N,N-dinonylformamide, N,N-didecylformamide, N,N-diundecylformamide, N,N-didodecylformamide, N,N-dihydroxymethylformamide, N,N-di-tert-butylacetamide, N,N-dipentylacetamide, N,N-dihexylacetamide, N,N-diheptylacetamide, N,N-dioctylacetamide, N,N-dinonylacetamide, N,N-didecylacetamide, N,N-diundecylacetamide, N,N-didodecylacetamide, N,N-dihydroxymethylacetamide, N,N-dimethylpropionamide, N,N-diethylpropionamide, N,N-dipropylpropionamide, such as N,N-di-n-propylpropionamide or N,N-diisopropylpropionamide, N,N-dibutylpropionamide, such as N,N-di-n-butylpropionamide, N,N-di-sec-butylpropionamide, N,N-diisobutylpropionamide or N,N-di-tert-butylpropionamide, N,N-dipentylpropionamide, N,N-dihexylpropionamide, N,N-diheptylpropionamide, N,N-dioctylpropionamide, N,N-dinonylpropionamide, N,N-didecylpropionamide, N,N-diundecylpropionami-de, N,N-didodecylpropionamide, N,N-dimethyl-n-butyramide, N,N-diethyl-n-butyramide, N,N-dipropyl-n-butyramide, such as N,N-di-n-propyl-n-butyramide or N,N-diisopropyl-n-butyramide, N,N-dibutyl-n-butyramide, such as N,N-di-n-butyl-n-butyramide, N,N-di-sec-butyl-n-butyramide, N,N-diisobutyl-n-butyramide, N,N-di-tert-butyl-n-butyramide, N,N-dipentyl-n-butyramide, N,N-dihexyl-n-butyramide, N,N-diheptyl-n-butyramide, N,N-dioctyl-n-butyramide, N,N-dinonyl-n-butyramide, N,N-didecyl-n-butyramide, N,N-diundecyl-n-butyramide, N,N-didodecyl-n-butyramide, N,N-dipentylisobutyramide, N,N-dihexylisobutyramide, N,N-diheptylisobutyramide, N,N-dioctylisobutyramide, N,N-dinonylisobutyramide, N,N-didecylisobutyramide, N,N-diundecylisobutyramide, N,N-didodecylisobutyramide, N,N-pentylhexylformamide, N,N-pentylhexylacetamide, N,N-pentylhexylpropionamide, N,N-pentylhexyl-n-butyramide, N,N-pentylhexylisobutyramide, N,N-methylethylpropionamide, N,N-methyl-n-propylpropionamide, N,N-methylisopropylpropionamide, N,N-methyl-n-butylpropionamide, N,N-methylethyl-n-butyramide, N,N-methyl-n-butyramide, N,N-methylisopropyl-n-butyramide, N,N-methyl-n-butyl-n-butyramide, N,N-methylethylisobutyramide, N,N-methyl-n-propylisobutyramide, N,N-methylisopropylisobutyramide, N,N-methyl-n-butylisobutyramide.

Surfactants present in the compositions according to the invention are, for example, nonaromatic-based surfactants, e.g. those based on heterocycles, olefins, aliphatics or cycloaliphatics, for example surface-active mono- or poly-alkyl-substituted and subsequently derivatized, e.g. alkoxylated, sulfated, sulfonated or phosphated, pyridine, pyrimidine, triazine, pyrole, pyrrolidine, furan, thiophene, benzoxazole, benzthiazole and triazole compounds, and/or aromatic-based surfactants, e.g. mono- or poly-alkyl-substituted and subsequently derivatized, e.g. alkoxylated, sulfated, sulfonated or phosphated, benzenes or phenols. The surfactants are generally soluble in the solvent phase and are preferably suitable for emulsifying it (together with active ingredients dissolved therein) upon dilution with water to give a spray liquor. The surfactant component when present in compositions according to the invention can, for example, comprise nonaromatic or aromatic surfactants or mixtures of nonaromatic and aromatic surfactants.

Examples of surfactants are listed below, in which EO represents ethylene oxide units, PO represents propylene oxide units and BO represents butylene oxide units:
1) C₁₀-C₂₄-alcohols which may be alkoxylated, e.g. with 1-60 alkylene oxide units, preferably 1-60 EO and/or 1-30 PO and/or 1-15 BO in any order. The terminal hydroxyl groups of these compounds can be terminally capped by an alkyl, cycloalkyl or acyl radical having 1-24 carbon atoms.
2) Anionic derivatives of the products described under b1) in the form of ether carboxylates, sulfonates, sulfates and phosphates and their inorganic salts (e.g. alkali metal and alkaline earth metal) and organic salts (e.g. those based on amine or alkanolamine).
3) Copolymers consisting of EO, PO and/or BO units, such as, for example, block copolymers with a molecular weight of from 400 to 10⁸.
4) Alkyleneoxy adducts of C₁-C₉ alcohols.
5) Fatty acid and triglyceride alkoxylates, or alkoxylated plant oils, such as soybean oil, rapeseed oil, corn oil, sunflower oil, cottonseed oil, linseed oil, coconut oil, palm oil, thistle oil, walnut oil, peanut oil, olive oil or rhicinus oil (i.e. castor oil), in particular rapeseed oil and castor oil, plant oils also being understood as meaning their transesterification products, e.g. alkyl esters, such as rapeseed oil methyl ester or rapeseed oil ethyl ester, salts of aliphatic, cycloaliphatic and olefinic carboxylic acids and polycarboxylic acids, and alpha-sulfo fatty acid esters.
6) Fatty acid amide alkoxylates.
7) Alkyleneoxy adducts of alkynediols.
8) Sugar derivatives, such as amino and amido sugars, glucitols, alkyl polyglycosides or such as sorbitan esters or cyclodextrine esters or ethers.
9) Surface-active cellulose and algine, pectin and guar derivatives.
10) Alkyleneoxy adducts based on polyol and interface-active polyglycerides and derivatives.
11) Sulfosuccinates, alkanesulfonates, paraffin- and olefin sulfonates.
12) Sulfosuccinamates.
13) Alkylene oxide adducts of fatty amines, quaternary ammonium compounds having 8 to 22 carbon atoms (C₈-C₂₂).
14) Surface-active zwitterionic compounds, such as taurides, betaines and sulfobetaines.
15) Surface-active compounds based on silicone and/or silane.
16) Per- or polyfluorinated surface-active compounds.
17) Interface-active sulfonamides.
18) Interface-active polyacrylic and polymethacrylic derivatives.
19) Surface-active polyamides, such as modified gelatin or derivatized polyaspartic acid and derivatives thereof.
20) Surface-active polyvinyl compounds, such as modified polyvinylpyrolidone, or the derivatized polyvinylacetates, or the butyrates, or modified polyvinyl alcohols.
21) Surface-active polymers based on maleic anhydride and/or reaction products of maleic anhydride, and maleic anhydride and/or reaction products of copolymers which include maleic anhydride.
22) Surface-active derivatives of montane, polyethylene and polypropylene waxes.
23) Surface-active phosphonates and phosphinates.
24) Poly- or perhalogenated surfactants.
25) Phenols which may be alkoxylated, for example phenyl (C₁-C₄)alkyl ethers or (poly)alkoxylated phenols [=phenol (poly)alkylene glycol ethers], for example having 1 to 50 alkyleneoxy units in the (poly)alkyleneoxy moiety, where the alkylene moiety preferably in each case has 1 to 4 carbon atoms, preferably phenol reacted with 3 to 10 mol of alkylene oxide, (poly)alkylphenols or (poly)alkylphenol alkoxylates [=polyalkylphenol (poly)alkylene glycol ethers], for example with 1 to 12 carbon atoms per alkyl radical and 1 to 150 alkyleneoxy units in the polyalkyleneoxy moiety, preferably tri-n-butylphenol or triisobutylphenol reacted with 1 to 50 mol of ethylene oxide, polyarylphenols or polyarylphenol alkoxylates [=polyarylphenol (poly)alkylene glycol ethers], for example tristyrylphenol polyalkylene glycol ethers with 1 to 150 alkyleneoxy units in the polyalkyleneoxy moiety, preferably tristyrylphenol reacted with 1 to 50 mol of ethylene oxide.
26) Compounds which formally represent the reaction products of the molecules described under 25) with sulfuric acid or phosphoric acid, and salts thereof neutralized with suitable bases, for example the acidic phosphoric esters of triethoxylated phenol, the acidic phosphoric ester of a nonylphenol reacted with 9 mol of ethylene oxide and the phosphoric ester of the reaction product of 20 mol of ethylene oxide and 1 mol of tristyrylphenol which has been neutralized with triethanolamine.
27) Benzenesulfonates, such as alkyl- or arylbenzenesulfonates, e.g. (poly)alkyl- and (poly)arylbenzenesulfonates which are acidic and neutralized with suitable bases, for example having 1 to 12 carbon atoms per alkyl radical or having up to 3 styrene units in the polyaryl radical, preferably (linear) dodecylbenzenesulfonic acid and oil-soluble salts thereof, such as, for example, the calcium salt or the isopropylammonium salt of dodecylbenzenesulfonic acid.

Examples of surfactants from the group of nonaromatic-based surfactants are the surfactants of the abovementioned groups 1) to 24), preferably the groups 1), 2), 11) and 13).

Examples of surfactants from the group of aromatic-based surfactants are the surfactants of the abovementioned groups 25)-27), preferably phenol reacted with 4 to 10 mol of ethylene oxide, triisobutylphenol reacted with 4 to 50 mol of ethylene oxide, nonylphenol reacted with 4 to 50 mol of ethylene oxide, tristyrylphenol reacted with 4 to 150 mol of ethylene oxide, and acidic (linear) dodecylbenzenesulfonate,

Preferred surfactants are, e.g. alkoxylated C₁₀-C₂₄-alcohols (1) and anionic derivatives thereof (2), such as sulfates, sulfonates and phosphates, alkoxylated plant oils (3), alkoxylated phenols (20) and reaction products thereof in sulfuric acid or phosphoric acid (21) and alkylbenzenesulfonates (22).

The weight ratio of solvent to surfactant is preferably in the range from 10 000:1 to 1:99, preferably from 1000:1 to 10:90, the solvent a) is particularly preferably in excess relative to the surfactant b), e.g. in the weight ratio from 100:1 to 2:1.

The composition of the invention preferably comprises in the range of from 0.5 to 30% by weight of the total composition of the surfactant component and more preferably from 2 to 20% by weight of the surfactant component.

The compositions of the invention are particularly suitable, for example, for the preparation of active ingredient formulations such as solution concentrates and emulsions concentrates suspensions, oil suspension concentrates, suspoemulsions, suspoemulsion concentrates, emulsions, e.g. W/O- or O/W-based ones, emulsion concentrates, microemulsions, microemulsion concentrates, and (aqueous) spray liquors obtainable therefrom.

The composition of the invention may include one or more phosphorylated alcohol ethoxylate which we have found is particularly useful in stabilizing the concentrate and also in stabilizing the diluted concentrate when admixed with a fertilizer such as urea-ammonium nitrate (UAN).

Examples of suitable phosphate ester surfactants include:
(i) alcohol phosphates such as alkyl mono and di-phosphates (eg. N-butyl mono/di-phosphate) and C₆ to C₁₀ alkyl phosphate esters;
(ii) alcohol ethoxylate phosphates such as C₈ to C₁₀ alcohol ethoxylate phosphate esters, tridecyl alcohol ethoxylate phosphate esters, C₁₀ to C₁₅ alcohol ethoxylate phosphate esters, butyl cellosolve phosphate esters, oleyl alcohol ethoxylate phosphate esters;
(iii) alkyl phenol ethoxylate phosphates such as nonylphenol ethoxylate phosphate ester and salts thereof and dinonylphenol ethoxylate phosphate ester;
(iv) (iv) aryl phosphate ethoxylate esters such as phenol ethoxylate phosphate esters; and
(v) alkyl amine phosphates such as triethylammonium phosphate.

A particularly preferred surfactant of this class is Teric® 305 alkyl ester phosphate based surfactant.

The composition of the invention may comprise one or more additional herbicides. Additional herbicides may be selected from the active ingredients listed in Table 1

TABLE 1

| Chemical Family | Active Ingredient |
|---|---|
| Aryloxyphenoxy- | clodinafop-propargyl |
| propionate | cyhalofop-butyl |
| ‘FOPs’ | diclofop-methyl |
| | fenoxaprop-P-ethyl |
| | fluazifop-P-butyl |
| | haloxyfop-R-methyl |
| | propaquizafop |
| | quizalofop-P-ethyl |
| Cyclohexanedione | alloxydim |
| ‘DIMs’ | butroxydim |
| | clethodim |
| | cycloxydim |
| | profoxydim |
| | sethoxydim |
| | tepraloxydin |
| | tralkoxydim |
| Sulfonylurea | amidosulfuron |
| | azimsulfuron |
| | bensulfuron-methyl |
| | chlorimuron-ethyl |
| | chlorsulfuron |
| | cinosulfuron |
| | cyclosulfamuron |
| | ethametsulfuron- |
| | methyl |
| | ethoxysulfuron |
| | flazasulfuron |
| | flupyrsulfuron-methyl- |
| | Na |
| | foramsulfuron |
| | halosulfuron-methyl |
| | imazosulfuron |
| | iodosulfuron |
| | mesosulfuron |
| | metsulfuron-methyl |
| | nicosulfuron |
| | oxasulfuron |
| | primisulfuron-methyl |
| | prosulfuron |
| | pyrazosulfuron-ethyl |
| | rimsulfuron |
| | sulfometuron-methyl |
| | sulfosulfuron |
| | thifensulfuron-methyl |
| | triasulfuron |
| | tribenuron-methyl |
| | trifloxysulfuron |
| | triflusulfuron-methyl |
| | tritosulfuron |
| Sulfonylurea | amidosulfuron |
| | azimsulfuron |
| | bensulfuron-methyl |
| | chlorimuron-ethyl |
| | chlorsulfuron |
| | cinosulfuron |
| | cyclosulfamuron |
| | ethametsulfuron- |
| | methyl |
| | ethoxysulfuron |
| | flazasulfuron |
| | flupyrsulfuron-methyl- |
| | Na |
| | foramsulfuron |
| | halosulfuron-methyl |
| | imazosulfuron |
| | iodosulfuron |
| | mesosulfuron |
| | metsulfuron-methyl |
| | nicosulfuron |
| | oxasulfuron |
| | primisulfuron-methyl |
| | prosulfuron |
| | pyrazosulfuron-ethyl |
| | rimsulfuron |
| | sulfometuron-methyl |
| | sulfosulfuron |
| | thifensulfuron-methyl |
| | triasulfuron |
| | tribenuron-methyl |
| | trifloxysulfuron |
| | triflusulfuron-methyl |
| | tritosulfuron |
| Triazolopyrimidine | cloransulam-methyl |
| | diclosulam |
| | florasulam |
| | flumetsulam |
| | metosulam |
| | penoxsulam |
| Pyrimidinyl(thio)benzoate | bispyribac-Na |
| | pyribenzoxim |
| | pyriftalid |
| | pyrithiobac-Na |
| | pyriminobac-methyl |
| Sulfonylaminocarbonyl- | flucarbazone-Na |
| triazolinone | propoxycarbazone-Na |
| Triazine | ametryne |
| | atrazine |
| | cyanazine |
| | desmetryne |
| | dimethametryne |
| | prometon |
| | prometryne |
| | propazine |
| | simazine |
| | simetryne |
| | terbumeton |
| | terbuthylazine |
| | terbutryne |
| | trietazine |
| Triazinone | hexazinone |
| | metamitron |
| | metribuzin |
| Triazolinone | amicarbazone |
| Uracil | bromacil |
| | lenacil |
| | terbacil |
| Pyridazinone | pyrazon = chloridazon |
| Phenyl-carbamate | desmedipham |
| | phenmedipham |
| Urea | chlorobromuron |
| | chlorotoluron |
| | chloroxuron |
| | dimefuron |
| | diuron |
| | ethidimuron |
| | fenuron |
| | fluometuron |
| | isoproturon |
| | isouron |
| | linuron |
| | methabenzthiazuron |
| | metobromuron |
| | metoxuron |
| | monolinuron |
| | neburon |
| | siduron |
| | tebuthiuron |
| Amide | propanil |
| | pentanochlor |
| Nitrile | bromofenoxim |
| | bromoxynil |
| | ioxynil |
| Benzothiadiazinone | bentazon |
| Phenyl-pyridazine | pyridate |
| | pyridafol |
| Bipyridylium | diquat |
| | paraquat |
| Diphenylether | acifluorfen-Na |
| | bifenox |
| | chlomethoxyfen |
| | fluoroglycofen-ethyl |
| | fomesafen |
| | halosafen |
| | lactofen |
| | oxyfluorfen |
| Phenylpyrazole | fluazolate |
| | pyraflufen-ethyl |
| N-phenylphthalimide | cinidon-ethyl |
| | flumioxazin |
| | flumiclorac-pentyl |
| Thiadiazole | fluthiacet-methyl |
| | thidiazimin |
| Oxadiazole | oxadiazon |
| | oxadiargyl |
| Triazolinone | azafenidin |
| | carfentrazone-ethyl |
| | sulfentrazone |
| Oxazolidinedione | pentoxazone |
| Pyrimidindione | benzfendizone |
| | butafenacil |
| Other | pyraclonil |
| | profluazol |
| | flufenpyr-ethyl |
| Pyridazinone | norflurazon |
| Pyridinecarboxamide | diflufenican |
| | picolinafen |
| Other | beflubutamid |
| | fluridone |
| | flurochloridone |
| | flurtamone |
| Triketone | mesotrione |
| | sulcotrione |
| Isoxazole | isoxachlortole |
| | isoxaflutole |
| Pyrazole | benzofenap |
| | pyrazolynate |
| | pyrazoxyfen |
| Other | benzobicyclon |
| Triazole | amitrole |
| | (in vivo inhibition of |
| | lycopene cyclase) |
| Isoxazolidinone | clomazone |
| Urea | fluometuron (see C2) |
| Diphenylether | aclonifen |
| Glycine | glyphosate |
| | sulfosate |
| Phosphinic acid | glufosinate-ammonium |
| | bialaphos = |
| | bilanaphos |
| Carbamate | asulam |
| Dinitroaniline | benefin = benfluralin |
| | butralin |
| | dinitramine |
| | ethalfluralin |
| | oryzalin |
| | pendimethalin |
| | trifluralin |
| Phosphoroamidate | amiprophos-methyl |
| | butamiphos |
| Pyridine | dithiopyr |
| | thiazopyr |
| Benzamide | propyzamide = |
| | pronamide |
| | tebutam |
| Benzoic acid | DCPA = chlorthal- |
| | dimethyl |
| Carbamate | chlorpropham |
| | propham |
| | carbetamide |
| Chloroacetamide | acetochlor |
| | alachlor |
| | butachlor |
| | dimethachlor |
| | dimethanamid |
| | metazachlor |
| | metolachlor |
| | pethoxamid |
| | pretilachlor |
| | propachlor |
| | propisochlor |
| | thenylchlor |
| Acetamide | diphenamid |
| | napropamide |
| | naproanilide |
| Oxyacetamide | flufenacet |
| | mefenacet |
| Tetrazolinone | fentrazamide |
| Other | anilofos |
| | cafenstrole |
| | piperophos |
| Nitrile | dichlobenil |
| | chlorthiamid |
| Benzamide | isoxaben |
| Triazolocarboxamide | flupoxam |
| Quinoline carboxylic acid | quinclorac |
| Dinitrophenol | DNOC |
| | dinoseb |
| | dinoterb |
| Thiocarbamate | butylate |
| | cycloate |
| | dimepiperate |
| | EPTC |
| | esprocarb |
| | molinate |
| | orbencarb |
| | pebulate |
| | prosulfocarb |
| | thiobencarb = |
| | benthiocarb |
| | tiocarbazil |
| | triallate |
| | vernolate |
| Phosphorodithioate | bensulide |
| Benzofuran | benfuresate |
| | ethofumesate |
| Chloro-Carbonic-acid | TCA |
| | dalapon |
| | flupropanate |
| Phenoxy-carboxylic-acid | clomeprop |
| | 2,4-D |
| | 2,4-DB |
| | dichlorprop = 2,4-DP |
| | MCPA |
| | MCPB |
| | mecoprop = MCPP = |
| | CMPP |
| Benzoic acid | chloramben |
| | dicamba |
| | TBA |
| Pyridine carboxylic acid | clopyralid |
| | fluroxypyr |
| | picloram |
| | triclopyr |
| | aminopyralid |
| Quinoline carboxylic acid | quinclorac |
| | quinmerac |
| Other | benazolin-ethyl |
| Phthalamate | naptalam |
| Semicarbazone | diflufenzopyr-Na |
| Arylaminopropionic acid | Flamprop-M-methyl/- |
| | isopropyl |
| Pyrazolium | difenzoquat |
| Organoarsenical | DSMA |
| | MSMA |
| Other | bromobutide |
| | (chloro)-flurenol |
| | cinmethylin |
| | cumyluron |
| | dazomet |
| | dymron = daimuron |
| | methyl-dimuron = |
| | methyl-dymron |
| | etobenzanid |
| | fosamine |
| | indanofan |
| | metam |
| | oxaziclomefone |
| | oleic acid |
| | pelargonic acid |
| | pyributicarb |

In a further aspect, the present invention provides a method of controlling weeds comprising applying a herbicidally effective amount of a herbicidal composition or a ready to use herbicidal composition as described herein to an area of land comprising weeds and/or in which pre-emergent control is desired.

Liquid compositions of the invention may be applied by spraying, atomizing, watering, introduction into the irrigation water, or any other suitable means for broadcasting or spreading the liquid.

The rate of application of the compositions of the invention will depend on a number of factors including, for example, the compound chosen for use, the identity of the plants whose growth is to be inhibited, the formulations selected for use, whether the compound is to be applied for pre-emergent or post-emergent control and whether for foliage or root uptake. As a general guide, however, an application rate of from 0.01 to 20 kilograms of compound of formula I per hectare (based on aromatic acid equivalent) is suitable while from 0.1 to 10.0 kilograms of the may be preferred.

The compounds of formula I may be prepared by a range of esterification methods generally known in the art. Trans-esterification may be used to prepare compounds of formula I which are a monoester of the group of formula I by reacting a methyl or ethyl ester of the aromatic acid with alcohol such as glycerol in the presence of a base catalyst such as potassium hydroxide. The reaction may be carried out in excess glycerol and at an elevated temperature of for example 100° to 200° C. under an inert atmosphere. Alternatively and more preferably esterification occurs by reaction between the phenoxy acid and glycerol preferably in the presence of a suitable catalyst such as an acid catalyst (e.g. an aromatic sulfonic acid) or base catalyst (e.g. potassium hydroxide or sodium methoxide). The reaction is preferably conducted in an inert solvent such as an alkyl benzene of flash point over 60.5° C.

The stoichiometry and reaction conditions may be used to control the relative preoprion of mono-, di- and tri-ester products.

The present invention is described with reference to the following examples. It is to be understood that the examples are illustrative of and not limiting to the invention described herein.

### EXAMPLES

### Example 1

### Synthesis of Tri-(2,4D)-ester of Glycerol

A 1 I four neck round bottom flask equipped with Dean-Stark adapter and thermometer was charged with 442.7 g 2,4D acid (2 mol), 200 g of Toluene, 61.9 g Glycerol (0.67 mol) and para-toluenesulfonic acid (pTSA) 3.16 g.

The mixture kept under reflux until no more water collected in the Dean-Stark adapter. This takes about 2 hrs. The water distilled as Water-Toluene azeotrop 37 mL. Analysis shows 14.96% of free acid.

6 g of Glycerol was added, and free acid dropped in another hour to 8.88%.

3 Glycerol additions were done more to drop free acid up to 4.41%. Total Glycerol charge was 82.4 g (0.896 mol). Excess of Glycerol probably distilled off with water.

Total water distilled 40 mL.

Toluene was stripped off from the reaction mixture under vacuum. Final temperature 160° C. Vacuum 30 mmbar (estimated).

Mass of product 466 g. Product appeared as a mixture of esters containing mainly the tri-ester.

Chemical shifts determined by nuclear magnetic resonance spectroscopy (NMR) are shown in the following table, where “m” indicates a multiplet peak, “s” indicates a singlet peak and “dd” indicates a “doublet doublet” peak

| Peaks | Chemical Shifts |
|---|---|
| m | 3.35 |
| m | 3.56 |
| m | 3.68 |
| m | 3.95 |
| dd | 4.14 |
| dd | 4.32 |
| dd | 4.43 |
| s | 4.84 |
| s | 4.97 |
| m | 5.09 |
| m | 5.34 |
| m | 7.1 |
| dd | 7.32 |
| m | 7.58 |

The following composition was determined by NMR

| | |
|---|---|
| Glycerol ester of 1-mono-(2,4D) | 7.9% |
| Glycerol ester of 1,2-di-(2,4D) | 16.1% |
| Glycerol ester of 1,2,3-tri-(2,4D) | 49.3% |
| Glycerol ester of 2-mono-(2,4D) | 19.1% |
| Glycerol ester of 1,3-di-(2,4D) | 4.6% |
| Free Glycerol | 2.2% |
| Toluene | 0.2% |
| Free 2,4D | not reported |

### Example 2

### Synthesis of Tetra (2,4D) ester of Pentaerythritol

A 1 L four neck round bottom flask equipped with Dean-Stark adapter and thermometer was charged with 442.7 g 2,4D acid (2 mol), 200 g of toluene, 69.1 g pentaerythritol (0.508 mol) and para-toluenesulfonic acid 3.0 g.

The mixture kept under reflux until no more water collected in the Dean-Stark adapter. This takes about 2 hrs. The water was distilled as the water-toluene azeotrope 36 mL. Analysis shows 1.13% of free acid.

Toluene was stripped off from the reaction mixture under vacuum. The final temperature was 160° C. Vacuum 30 mmbar (estimated).

Mass of product 475 g. The product was a light coloured crystal.

Chemical shifts determined by nuclear magnetic resonance spectroscopy (NMR) are shown in the following table, where “m” indicates a multiplet peak, “s” indicates a singlet peak and “dd” indicates a “doublet doublet” peak

| Peaks | Chemical Shifts |
|---|---|
| s | 2.3 |
| s | 3.34 |
| s | 4.16 |
| s | 4.72 |
| s | 4.83 |
| s | 4.98 |
| d | 7.07 |
| dd | 7.26 |
| d | 7.56 |

The following composition was determined by carbon and proton NMR

| | |
|---|---|
| Tetrakis 2,4-D ester of pentaerythritol | 94.3% |
| Tris 2,4D ester of pentaerythritol | 3.1% |
| Free 2,4D | 1.7% |
| Toluene | 0.9% |

### Example 3

### Tetra (Mecoprop-P) Ester of Pentaerythritol

The procedure of Example 2 was repeated accept that 2,4-D acid was replaced with the corresponding molar amount of Mecoprop-P.

The product was examined by carbon and proton NMR and found to contain the following components:

| | |
|---|---|
| Tetrakis Mecoprop-P ester of pentaerythritol | 87.6% w/w |
| Tris Mecoprop-P ester of pentaerythritol | 4.9% w/w |
| Mecoprop-P | 2.9% w/w |
| Toluene | 3.8% w/w |
| pTSA | 0.6% w/w |
| Others | <0.5% w/w |

### Example 4

The procedure of Example 2 was repeated except that 2,4-D acid was replaced with MCPA.

The product was examined by carbon and proton NMR and found to contain the following components:

| | |
|---|---|
| Tetrakis MCPA ester of pentaerythritol | 87.9% w/w |
| Tris MCPA ester of pentaerythritol | 4.9% w/w |
| MCPA | 3.0% w/w |
| Toluene | 3.1% w/w |
| pTSA | 0.8% w/w |
| Others | <0.5% w/w |

### Example 5

### Sample Preparation

The 2,4-D glycerol ester of Example 1 (0.483 g/100 mL) and TERMUL 5459 (Huntsman) (1.0 g/100 mL) were dissolved in JEFFSOL AG-1540. TERMUL 5459 is a (C₁₂-C₁₈) alkyl alcohol ethoxylate propoxylate emulsifier supplied by Huntsman. JEFFSOL AG-1540 is an alkylene carbonate solvent supplied by Huntsman.

### Plant Propagation

Canola (Brassica napus) and variegated thistle (Silybum marianum) were sown to 1 cm depth in 10 cm diameter pots filled with potting mix (AS 3743). One week after seedling emergence, seedlings were thinned for uniform size to one seedling per pot. Canola were germinated in a temperature-controlled greenhouse (14° C.-25° C.) for 7 days then outdoors for 10 days prior to spray application, to more closely simulate field conditions. Variegated thistle was germinated in a temperature-controlled greenhouse (14° C.-25° C.) for 7 days then outdoors for 17 days prior to spray application. After the application of herbicides the pots were returned to the greenhouse until harvested for fresh weight.

### Herbicide Application

Herbicide formulations were applied using an enclosed laboratory track-sprayer fitted with a 110° flat fan nozzle (Teejet XR11001-VS), at a pressure of 100 kPa applying a spray volume of 145 L/ha.

### Assessment

Seedlings were harvested by cutting foliage off at base immediately prior to weighing on a Sartorious Basic electronic balance (range 0-4100 g) 24DAT.

| | Canola | | Variegated Thistle | |
|---|---|---|---|---|
| Dose | Brassica napus | | Silybum marianum | |
| g 2,4-D/ha | mean | standard error | mean | standard error |
| 0 | 18.85 | 1.23 | 28.85 | 1.08 |
| 50 | 15.65 | 0.79 | 28.88 | 1.79 |
| 100 | 14074 | 0.89 | 30.21 | 1.29 |
| 200 | 7.39 | 0.78 | 24.10 | 1.57 |
| 300 | 7.17 | 1.23 | 21.04 | 1.76 |
| 400 | 7.15 | 0.38 | 24.31 | 1.24 |
| 600 | 6.06 | | 20.40 | 0.97 |

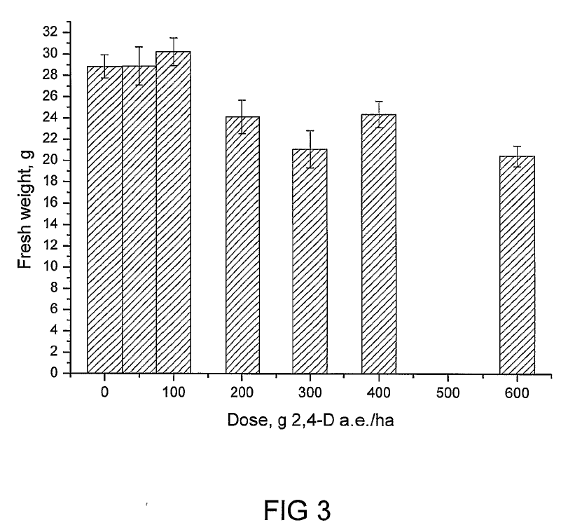

The results listed in the table are shown in the column charts in FIG. 2 for treatment of Canola Brassica napus and FIG. 3 for Variegated Thistle Silybum marianum.

### Example 6

### Suspension Concentrate (SC) Formulation Details

A suspension concentrate of the a phenoxy ester of the invention such as the di-(2,4-dichlorophenoxyacetate)glyceride or tetra-(2,4-dichlorophenoxyacetate)-pentaerythritol may be prepared from the following constituents in the amounts specified.

TABLE 2

| | | |
|---|---|---|
| Phenoxy Ester Technical | 300-800 | Active ingredient |
| (E.g. Tetrakis 2,4-D ester of | | |
| Pentaerythritol) | | |
| Propylene glycol | 60 ± 6 | Humectant/anti-freeze |
| Glycerine | 40 ± 4 | Humectant/anti-freeze |
| TERSPERSE 2500 (Huntsman) | 25 ± 3 | Surfactant |
| TERSPERSE 4894 (Huntsman) | 15 ± 2 | Surfactant |
| Xanthan Gum | 1.13 ± 0.1 | Viscosity modifier |
| eg RHODOPOL 23 (Rhodia) | | |
| 20% solution of | 0.25 ± 0.03 | Biocide |
| 1,2-benzisothiazolin-3-one | | |
| in dipropylene glycol | | |
| eg PROXEL GXL | | |
| Polydimethylsiloxane | 6 ± 0.6 | Antifoam |
| ANTIFOAM C (Dow Corning) | | |
| Water | To 1 Litre | Solvent |

The process steps may be summarised as follows:

### Preparation of Premix

Charge the a clean and dry vessel with the water, add the PROXEL GXL and RHODOPOL 23 and stir vigorously without air entrapment until all the RHODOPOL 23 has swelled.

### Preparation of Slurry

Charge with water and commence stirring.
Charge, in order:
PROPYLENE GLYCOL
GLYCERINE
TERSPERSE 4894
TERSPERSE 2500
ANTIFOAM C
Charge the PHENOXY ESTER TECHNICAL, allowing incorporation into the slurry to obtain a homogeneous mix. Recirculate via Silverson in-line mixer during charging.
When all the technical has been added continue stirring for 15 minutes.
Transfer slurry to T2 through the in-line Silverson mixer and maintain slow agitation.

### Milling of Slurry

Charge mill with 295 kg of ceramic beads, ie 65% of mill volume.
Commence milling on recycle until the required particle size is obtained then direct slurry flow to a finished product tank (T3/T4). Ensure milling temp does not exceed 36-40 degrees.
If temperature rises above 38 degrees for more than 5 minutess, or the current drag is near the maximum rating, as a first step stop the mill and remove 25 kg of beads. Start milling again and repeat if necessary. Sample continually to check particle size.
If the milling temp is stable at about 35 degrees and current draw is comfortably below the maximum rating, add 25 kg more beads and/or increase the feed rate and monitor particle size.

### Viscosity Adjustment

When the batch is fully transferred, charge:
PREMIX [see above]
Stir until both components are fully incorporated.

### Example 7

Water dispersible granules of the a phenoxy ester of the invention such as the tri-(2,4-dichlorophenoxyacetate)glyceride or tetra-(2,4-dichlorophenoxyacetate) pentaerythritol may be prepared from the following constituents in the amounts specified.

TABLE 3
Water Dispersible Granule (WDG) Formulation Details

| | | |
|---|---|---|
| Phenoxy Ester Technical | 50-92 | Active |
| (E.g. Tetrakis 2,4-D ester of Pentaerythritol) | | ingredient |
| TERSPERSE 2600 (Huntsman) | 3-5 | Dispersant |
| TERWET 1004 (Huntsman) | 1-3 | Wetter |
| talc | 1-40 | Inert filler |
| Residual Water | 0.5-1.0 | Binder |
| | (after drying) | |

### Preparation Process

### STAGE 1: Blending of Solid Materials

The active, TERSPERSE 2600, TERWET 1004 & talc are intimately mixed together making certain that a homogenous mixture is obtained.
A ribbon blender can be used.

### STAGE 2: Addition of Water

The water is added slowly in small amounts and homogenously mixed with the solid mixture. The amount of water added to the mixture is very critical to ensure that the extrusion step is made easier.
Generally, depending on the technical source of the active and the type of extruder used, approximately 15% w/w of water will be sufficient to provide a mixture that can produce granules that extrude easily and disperse fully.

### STAGE 3: Extrusion

A “basket” extruder is used to compact the homogenous moistened mixture through a screen, resulting in granules of the required shape & diameter.

### STAGE 4: Drying

The granules are dried on a fluid-bed drier at around 55° C.-60° C.
The ideal residual water content should be approximately 0.2-0.8% w/w.
Collect a sample of the finished.

### Example 8

Wettable powders of the a phenoxy ester of the invention such as the tri-(2,4-dichlorophenoxyacetate)glyceride or tetra-(2,4-dichlorophenoxyacetate) pentaerythritol may be prepared from the following constituents in the amounts specified.

TABLE 4
Wettable Powders (WP) Formulation Details

| | | |
|---|---|---|
| Phenoxy Ester Technical | 30-92 | Active |
| (E.g. Tetrakis 2,4-D ester of Pentaerythritol) | | ingredient |
| TERSPERSE 2600 (Huntsman) | 3-5 | Dispersant |
| TERIC 157 or TERWET 1004 | 1-3 | Wetter |
| (Huntsman) | | |
| talc | To 100% | Inert filler |

### Preparation Process

The active ingredient and all the other materials are mixed so as to achieve a homogenous mixture. A ribbon blender can be used to obtain the homogenous mixture. A dry mill is used to co-mill all the materials together so as achieve a homogenous mixture.

### Example 9

Using equations presented by Woodrow, Seiber and Baker in the journal Environmental Science & Technology (1997, Volume 31, issue 2 at page 523), the volatile loss (pesticide flux, Q) from various esters of the phenoxy herbicides 2,4-D (2,4-dichlorophenoxyacetic acid) and MCPA (4-chloro-2-methylphenoxyacetic acid) are shown in shown in the Table.

TABLE 5

| Phenoxy | Ester | log Kow | VP torr | VP Pa | Koc | SW | plant Flux Q | soil Flux Q |
|---|---|---|---|---|---|---|---|---|
| 2,4-D | ethyl | 3.39 | 4.98E−04 | 5.00E−04 | 2.90E+02 | 8.02E+01 | 200.18 | 0.90 |
| 2,4-D | 2-ethylhexyl | 6.27 | 4.57E−06 | 6.63E−04 | 1.05E+04 | 3.47E−02 | 254.52 | 1089.03 |
| 2,4-D | glycerol monoester | 1.29 | 6.30E−09 | 9.14E−07 | 1.00E+01 | 1.04E+03 | 9.42E−01 | 1.36E−04 |
| 2,4-D | glycerol diester | 4.42 | 5.12E−13 | 7.43E−11 | 5.91E+02 | 4.01E−01 | 3.15E−04 | 1.64E−08 |
| 2,4-D | glycerol triester | 7.95 | 2.01E−15 | 2.92E−13 | 2.24E+07 | 5.55E−06 | 2.83E−06 | 6.51E−12 |
| 2,4-D | pentaerythritol tetra-ester | 10.86 | 1.40E−18 | 1.86E−16 | 4.20E+09 | 9.30E−07 | 5.45E−09 | 2.02E−19 |
| MCPA | pentaerythritol tetra-ester | 10.47 | 4.32E−17 | 5.76E−15 | 4.16E+09 | 8.51E−07 | 1.01E−07 | 5.76E−17 |
| MCPA | glycerol tri-ester | 7.66 | 6.17E−12 | 8.23E−10 | 2.24E+07 | 7.13E−06 | 2.43E−03 | 1.44E−06 |
| MCPA | 1,2,4-butanetriol tri-ester | 8.15 | 3.05E−12 | 4.07E−10 | 4.13E+07 | 2.12E−06 | 1.33E−03 | 1.23E−06 |

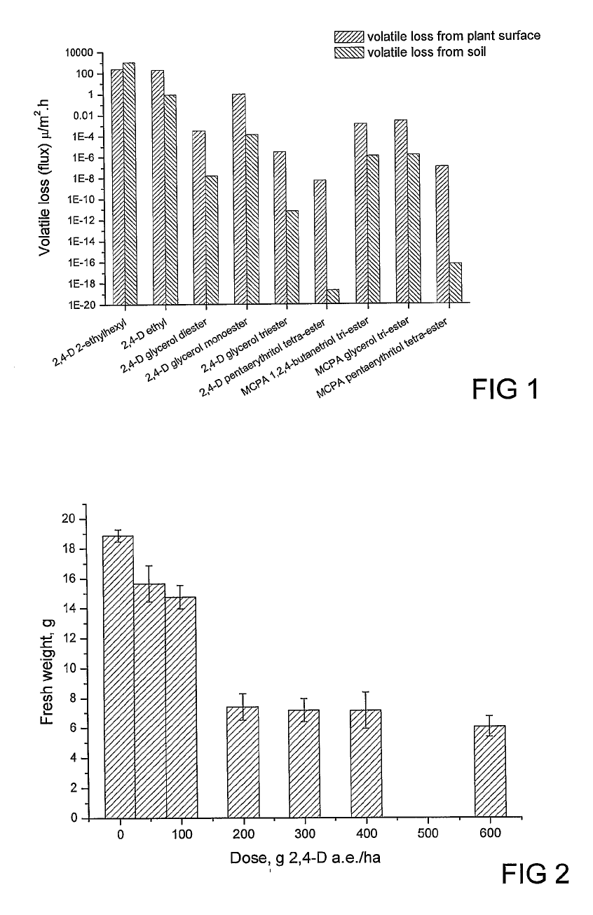

The volatile loss (pesticide flux, Q) for selected esters of phenoxy herbicides, following Woodrow, Seiber and Baker (1997). The results are plotted in FIG. 1.

The work of Woodrow, Seiber and Baker provides empirical correlations to estimate the flux (loss per unit area in unit time) of pesticides from plant surfaces, soil and water. Flux from plant surfaces was found to be a function of vapour pressure. Flux from soil was found to a function of the vapour pressure, soil absorption coefficient (K_{oc}) and water solubility (S_{w}), while loss from water was a function of vapour pressure and water solubility.

Plant surfaces are assumed to be inert with respect to interactions with the pesticide. The volatile loss from plant surfaces is estimated by Equation 1.

ln(Q)=11.76+0.85 ln(VP)

Equation 1: Volatile Loss from Plant Surfaces

Pesticide may absorb onto the soil or dissolve in water in the soil. The volatile loss from soils is estimated from Equation 2.

$$ln ( Q ) = 28.14 + 1.60 ln ( \frac{VP}{{K}_{oc} {S}_{w}} )$$

Equation 2: Volatile Loss from Soils

The volatile loss from water is estimated from Equation 3.

$$ln ( \frac{Q}{c} ) = 13.39 + 0.86 ln ( \frac{VP}{{S}_{w}} )$$

Equation 3: Volatile Loss from Water

### Where:

c is concentration of pesticide in water
Q is pesticide flux (μg/m²·h),
VP is vapour pressure (Pa),
K_{oc} is soil absorption coefficient (mL/g), and
S_{w} is water solubility (mg/L)

Finally, it is understood that various other modifications and/or alterations may be made without departing from the spirit of the present invention as outlined herein.

1. A herbicidal composition comprising at least one compound of formula I 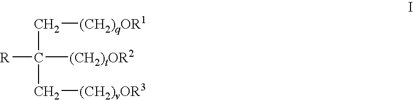
wherein
R is independently selected from hydrogen, and the group (CH₂)ₘOR⁴;
m, q, t and v (which are each independently selected) are each zero or one;
R¹, R², R³ and R⁴ are independently selected from the group consisting of hydrogen, lower alkyl and lower acyl and the group of formula II wherein the compound of formula I comprises at least one group R¹, R², R³ and R⁴ which is of formula II 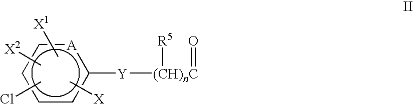
wherein
A is N or CH;
X is selected from the group consisting of chloro, amino, methyl and methoxy;
X¹ is selected from the group consisting of hydrogen, chloro and methyl;
X² is hydrogen or chloro;
R⁵ is selected from hydrogen and methyl;
Y is oxygen or a bond; and
n is from 0 to 3.

2. A herbicidal composition according to claim 1 wherein formula II complies with formula IIa 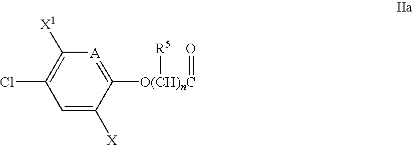
wherein
A is N or CH;
X is selected from the group of halogen and methyl;
X¹ is selected from hydrogen, halogen and methyl; and
n is from 1 to 3.

3. (canceled)

4. A composition according to claim 2 wherein the group of formula II is derived from a herbicidal acid selected from the group consisting of:
2,4-D (2,4-dichlorophenoxyacetic acid),
2,4-DB 2-(2,4-dichlorophenoxy)butyric acid,
dichlorprop-P (R)-2-(2,4-dichlorophenoxy)propionic acid,
MCPA 4-chloro-o-tolyloxyacetic acid,
MCPB 4-(4-chloro-O-tolyloxy)butyric acid, and
Mecoprop-P (R)-2-(4-chloro-o-tolyloxy)butyric acid.

5. A composition according to claim 2 wherein in the compound of formula I at least one of m, q, t and v is one

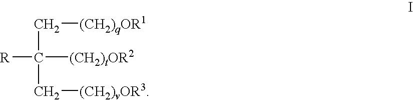

6. A composition according to claim 2 wherein the compound is derived from an alcohol selected from the group consisting of glycerol, butan-1,2,4-triol, 2-(hydroxymethyl)butan-1,4-diol, 3-(hydroxymethyl)pentan-1,5-diol, pentan-1,3,5-triol, 2-(hydroxymethyl)propan-1,2,3-triol, 2-(hydroxymethyl)butan-1,2,4-triol, 2,2-bis(hydroxymethyl)butan 1,4-diol, 3,3-bis(hydroxymethyl)pentan-1,5-diol, 3-(hydroxylmethyl)pentan-1,3,5-triol and pentaerythritol.

7. A composition according to claim 1 wherein the compound of formula I is prepared by a process comprising reacting an alcohol of formula I wherein R¹, R², R³ and R⁴ are selected from the group consisting of hydrogen, lower alkyl, lower acyl and at least one of R¹, R², R³ and R⁴ present in the compound of formula I is hydrogen with an acid or derivative thereof of formula V:
Y—R⁶ V
wherein Y is the group of formula II and R⁶ is —OH or a leaving group optionally in the presence of a catalyst to provide a compound of formula I wherein at least one of R¹, R², R³ and R⁴ is of formula IIa.

8. (canceled)

9. A herbicidal composition comprising at least one compound of formula I 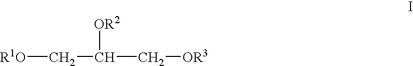
wherein at least one of R¹, R² and R³ is a group of formula II 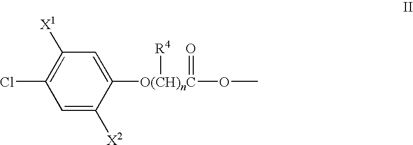
wherein
X¹ is selected from the group consisting of hydrogen, chloro and methyl;
X² is selected from the group consisting of hydrogen, chloro and methyl;
R⁴ is selected from hydrogen and methyl; and
n is from 1 to 3;
wherein the groups R¹, R² and R³ which are not of formula II are hydrogen.

10. A composition according to claim 9 wherein formula I is the mono glyceride, diglyceride, triglyceride of the phenoxy acid group or a mixture of two or more of the mono, di and triglycerides.

11. A composition according to claim 10 wherein the phenoxy acid is selected from the group consisting of
2,4-D (2,4-dichlorophenoxyacetic acid),
2,4-DB 2-(2,4-dichlorophenoxy)butyric acid,
dichlorprop-P (R)-2-(2,4-dichlorophenoxy)propionic acid,
MCPA 4-chloro-o-tolyloxyacetic acid,
MCPB 4-(4-chloro-O-tolyloxy)butyric acid, and
mecoprop-P (R)-2-(4-chloro-o-tolyloxy)butyric acid.

12. A composition according to claim 2 in the form of a concentrate for dilution with water or in the form of a dilute composition ready for application to plants.

13. The composition of the invention in the form of a concentrate containing at least 400 g/L based on corresponding acid equivalent of at least one compound of formula I as defined in claim 1.

14. A herbicidal composition according to claim 2 comprising:
(i) one or more esters of formula I,
(ii) a non-volatile solvent,
(iii) at least one surfactant, and
(iv) optionally customary auxiliaries and additives, such as thickeners and thixotropic agents, wetting agents, anti-drift agents, adhesives, penetration agents, preservatives and frost protection agents, antioxidants, fillers, carrier substances, dyes, fragrances, antifoams, fertilizers, evaporation inhibitors, and agents which influence the pH and the viscosity.

15. (canceled)

16. A herbicidal composition according to claim 2 in the form of an emulsifiable concentrate which comprises 0.05 to 90% of compound(s) of formula I, from 0.1 to 20% surfactant, and from 39 to 98.99%, of organic solvent.

17. A composition according to claim 4 wherein the compound of formula I is selected from the group consisting of the mono-, di-, tri- and tetra-2,4-D esters and the mono-, di-, tri- and tetra MCPA esters.

18. (canceled)

19. (canceled)

20. (canceled)

21. A composition according to claim 14 wherein the non-volatile solvent comprises at least one hydrocarbon selected from alkyl substituted aromatics such as mono-, di- and trialkyl benzenes and alkyl naphthalenes and preferably C₈-C₁₂ di- and trialkyl benzenes.

22. (canceled)

23. (canceled)

24. (canceled)

25. (canceled)

26. (canceled)

27. (canceled)

28. (canceled)

29. (canceled)

30. A composition according to claim 14 wherein the composition further comprises glyphosate.

31. A compound of formula III 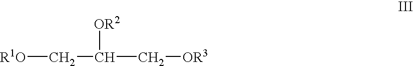
wherein at least two of R¹, R² and R³ is a group of formula IV 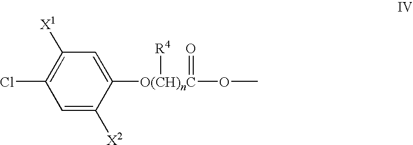
wherein
X¹ is selected from hydrogen, halogen (preferably chloro) and methyl, preferably from hydrogen and chloro and most preferably is hydrogen;
X² is selected from the group of hydrogen, halogen and methyl;
R⁴ is selected from hydrogen and methyl and preferably is hydrogen; and
n is from 1 to 3;
wherein the groups R¹, R² and R³ which are not of formula II are hydrogen and lower aliphatic.

32. A compound of formula I 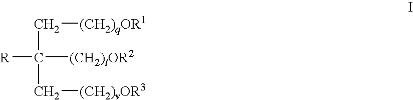
wherein
R is independently selected from hydrogen, and the group (CH₂)ₘOR⁴;
m, q, t and v and independently zero or one;
R¹, R², R³ and R⁴ are independently selected from the group consisting the group of hydrogen, lower alkyl and lower acyl and the group of formula II wherein the compound of formula I comprises at least one group R¹, R², R³ and R⁴ which is of formula II 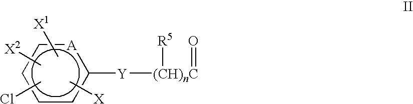
wherein
A is N or CH;
X is selected from the group consisting of chloro, amino, methyl and methoxy;
X¹ is selected from the group consisting of hydrogen, chloro and methyl;
X² is hydrogen or chloro;
R⁵ is selected from hydrogen and methyl;
Y is oxygen or a bond; and
n is from 0 to 3.

33. (canceled)

34. (canceled)

35. A composition according to claim 2 wherein the composition comprises at least one compound of formula I in solid particulate form.

36-41. (canceled)
